(19) United States
(12) Patent Application Publication
(10) Pub. No.: US 2005/0095209 A1
(43) Pub. Date: May 5, 2005

(54) Nail Application Containing Gotu Kola

(75) Inventor: Jacqueline Ann Lewchenko, Bartlett, IL (US)

Correspondence Address: SEYFARTH SHAW, 55 EAST MONROE STREET, SUITE 4200, CHICAGO, IL, 60603-5803, US

(73) Assignee: BE! PRODUCTS, INC., Mundelein, IL (US)

(21) Appl. No.: 10/605,859

(22) Filed: Oct. 31, 2003

(51) Int. Cl. A61K 7/04; A61K 7/135

(52) U.S. Cl. 424/61

(57) ABSTRACT

A nail application composition containing gotu kola. Gotu kola is applied to a nail to increase keratin production to strengthen and treat the nail. The gotu kola may be applied to the nail by a nail polish, nail lacquer, nail cream, nail oil, or other well known methods. When the gotu kola is applied to a nail, the wearer is able to simultaneously increase the nail's aesthetic appearance and increase keratin production for the nail.

### BACKGROUND OF INVENTION

The present application pertains generally to nail strengthening treatments and more specifically to a nail strengthening treatment containing gotu kola.

A nail is a corneous layer forming from the epidermis of the dorsal side of the fingers or toes. Nails are composed of various components, including the root or germinal matrix, nail bed or sterile matrix, nail plate, eponychium or cuticle, perionychium, and hyponychium. Nails enable us to seize objects by the tip of fingers and to carry out precise work. Nails are mostly composed of keratin, which is produced and secreted by keratinocytes or kertainized epithelial cells. A nail may become weak, thin, split, or peel for various reasons such as exposure to chemicals, water, or physical trauma. Nails are routinely painted and decorated to add aesthetic appeal.

It is well known that gotu kola, also known as Hydrocotyle asiatica, Centella asiatica, bramhi, hydrocotyle, and Indian Pennywort, can be used to treat dermal inflammation, aid in blood circulation, and aid in the treatment of bloating, congestion and depression. Gotu kola contains glycoside and asiatiocides which stimulate the formation of and aid in the strengthening of tissues, such as the dermis, nails, hairs, and other connective tissues. Gotu Kola also increases keratin and collagen synthesis.

Gotu kola contains at least three triterpenes, asiatic acid, madecassic acid, and asiaticoside. It is well known that asiaticoside, also known as centelase dermatologico and madecassol, stimulates the production of keratin, which, as described above, is essential to nail formation.

Gotu kola has previously been added to hair and skin treatments, such as soaps, shampoos, lotions, and facial scrubs. Hair and skin treatment ingredients are water soluble and hydrophilic. Nail application products, such as nail lacquers, nail oils, or nail polishes routinely use water insoluble hydrophobic materials. Gotu kola has relatively low solubility in hydrophobic solvents.

The compositions of nail applications, also known as nail lacquers and nail treatments, are well known in the art. In general, nail lacquers comprise a film former dissolved within a solvent. When the solvent evaporates, the film former forms a film like coating or deposit on a nail. Nail lacquers may also contain additional ingredients, such as, for example, plasticizers, ultraviolet radiation absorbers, pigments, viscosity controlling agents, and vitamins. It will be appreciated that a nail lacquer may have more than one film former, solvent, plasticizer, ultraviolet radiation absorber, pigment, and viscosity controlling agent, or various combinations thereof.

The film formers that are included in nail lacquers are well known in the art. Film formers are substances that create a substantially continuous film on a nail after application of the nail lacquer and substantial evaporation of the solvent. It is well known that such film formers may include resins, polymers, and cellulose materials.

Solvents used to dissolve the film former are also well known. Common solvents used to dissolve the film former in a nail lacquer may include alcohols, acetates, organic solvents, or inorganic solvents.

### DETAILED DESCRIPTION

In an embodiment, gotu kola is added to a nail application, such as a nail treatment or nail lacquer, to provide a nail strengthening composition. The gotu kola may be applied to the nail by a nail polish, nail lacquer, nail cream, nail oil, or other well known methods. When the gotu kola is applied to a nail, the wearer is able to simultaneously increase the nail's aesthetic appearance and increase keratin production for the nail.

Nail lacquers may also be generally described as nail polish, nail enamel, or nail basecoats. Nail lacquer is commonly applied to nails to increase a nail's aesthetic appearance. The addition of gotu kola to the nail application allows the wearer to simultaneously increase the strength of the wearer's nails due to gotu kola's natural keratin synthesis properties, as well as achieving the desired in-crease aesthetic appearance of a nail.

Gotu kola is water soluble. In an embodiment, the gotu kola is substantially dissolved in the nail application. In another embodiment, the gotu kola is partially dissolved in the nail application. In yet an embodiment, the gotu kola concentration in the composition is substantially in the range of 0.0001% -2% of the total weight of the composition. In yet another embodiment, the gotu kola concentration in the composition is substantially in the range of 0.00001% -2% of the total weight of the composition. In another embodiment, the gotu kola concentration in the composition is substantially 0.05% -0.5% of the total weight of the composition. In yet another embodiment, the gotu kola concentration in the composition is substantially 0.05% -0.5% of the total weight of the composition. In yet another embodiment, the nail application is a nail oil and gotu kola.

The present invention can be better illustrated with the following examples, which are intended to explain and not limit the invention.

Table 1 details the composition of one example of the present invention.

TABLE 1

| | Weight Percentage |
|---|---|
| Ingredients | Range |
| n-butyl acetate | 25-40% |
| Toluene | 20-35% |
| Nitrocellulose | 5-20% |
| ethyl acetate | 10-25% |
| tosylamide/formaldehyde resin | 5-20% |
| dibutyl phthalate | 1-10% |
| Isopropyl alcohol | 1-10% |
| Camphor-DL | 1-10% |
| Benzophenone-1 | .001-5% |
| gotu kola | .0001-2% |
| D&C violet #2 | .0001-2% |
| Vitamin B12 | .0001-2% |

The composition presented in Table 1 provides a formulation to produce a strengthened, fortified foundation for a generally weakened nail. Vitamin B12 is also included to reinforce the strength and flexibility of the nail.

Table 2 details the composition of another example of the present invention.

TABLE 2

| | Weight Percentage |
|---|---|
| Ingredients | Range |
| Toluene | 25-40% |
| ethyl acetate | 20-35% |
| n-butyl acetate | 5-20% |
| Nitrocellulose | 1-20% |
| tosylamide/formaldehyde resin | 1-20% |
| dibutyl phthalate | 1-15% |
| isopropyl alcohol | 1-15% |
| n-butyl alcohol | .001-3% |
| Formaldehyde | .001-3% |
| Etocrylene | .001-3% |
| gotu kola | .0001-2% |
| D&C violet #2 | .0001-2% |
| Vitamins, such as vitamin B5 | .0001-2% |

The composition in Table 2 provides for a formulation to reinforce and add strength to a ridged, weak and thin, or peeling nail. The film formers deposit a protective coating to a nail. The film formers assist the nail in resisting physical trauma.

Table 3 details the composition of another example of the present invention.

TABLE 3

| | Weight Percentage |
|---|---|
| Ingredients | Range |
| ethyl acetate | 20-40% |
| n-butyl acetate | 20-40% |
| isopropyl alcohol | 10-25% |
| Toluene | 5-15% |
| Phthalic anhydride/trimellitic anhydride/glycols | 1-10% |
| copolymer | |
| tosylamide/epoxy resin | 1-10% |
| Nitrocellulose | 1-10% |
| magnesium silicate | 1-10% |
| Hydrated silica | 1-10% |
| Nylon | .100-5% |
| Polyurethane | .100-5% |
| triphenyl phosphate | .100-5% |
| trimethyl pentanyl diisobutyrate | .100-5% |
| n-butyl alcohol | .001-3% |
| Camphor (DL) | .001-3% |
| Formaldehyde | .001-3% |
| stearalkonium hectorite | .001-3% |
| stearalkonium bentonite | .001-3% |
| citric acid | .0001-2% |
| gotu kola | .0001-2% |
| Hypnea musciformis | .0001-2% |
| D&C violet #2 | .0001-2% |

The composition in Table 3 provides for a formulation to reinforce and add strength to a ridged, weak and thin, or peeling nail. Nylon is provided as a viscosity controlling agent.

Table 4 details the composition of another example of the present invention.

TABLE 4

| | Weight Percentage |
|---|---|
| Ingredients | Range |
| n-butyl acetate | 25-40% |
| Toluene | 20-35% |
| ethyl acetate | 10-25% |
| Nitrocellulose | 5-20% |
| tosylamide/formaldehyde resin | 5-20% |
| dibutyl phthalate | 1-10% |
| isopropyl alcohol | 1-10% |
| n-butyl alcohol | .001-3% |
| Formaldehyde | .001-3% |
| camphor (DL) | .001-3% |
| Benzophenone-1 | .001-3% |
| gotu kola | .0001-2% |
| D&C violet #2 | .0001-2% |

The composition in Table 4 is a formulation which provides general maintenance to keratinocytes and a shield for pigment migration.

Table 5 details the composition of another example of the present invention.

TABLE 5

| | Weight Percentage |
|---|---|
| Ingredients | Range |
| n-butyl acetate | 25-40% |
| Toluene | 25-40% |
| ethyl acetate | 10-25% |
| Nitrocellulose | 5-20% |
| tosylamide/formaldehyde resin | 5-20% |
| dibutyl phthalate | 5-20% |
| isopropyl alcohol | 1-15% |
| n-butyl alcohol | .001-3% |
| Formaldehyde | .001-3% |
| camphor (DL) | .001-3% |
| Benzophenone-1 | .001-3% |
| gotu kola | .001-2% |
| D&C violet #2 | .0001-2% |
| hemp seed oil | .0001-2% |

The composition in Table 5 provides for a formulation created to apply to a weak and thin or soft nail to increasing keratin synthesis. The composition is also capable of rejuvenating and restoring a damaged nail by increasing keratin synthesis. The composition further aids the existing nail keratin from splitting, tearing, peeling or chipping, and contains hemp seed oil extract, which aids in the restructuring of keratinocytes.

It is understood that additional components or trace materials, contaminants, water and gases may also be present in the illustrative examples without affecting the composition's ability to be applied to nails or to form a film.

When a nail application in accordance with the present invention is applied to a nail, one or more solvents evaporate to form a substantially water insoluble film on the nail. It is preferred that a substantially water insoluble film be formed on the nail so as to prevent the flaking or washing off of the film with water or other hydrophilic materials. Accordingly, it is preferred that the film forming material be substantially water insoluble.

The matter set forth in the foregoing description and accompanying drawings is offered by way of illustration only and not as a limitation. While particular embodiments have been shown and described, it will be apparent to those skilled in the art that changes and modifications may be made without departing from the broader aspects of applicants” contribution. The actual scope of the protection sought is intended to be defined in the following claims when viewed in their proper perspective based on the prior art.

1. A composition having a total weight for topical application on a nail comprising:
gotu kola and a film former, the gotu kola and film former being dissolved in a solvent that is capable of evaporating to deposit the gotu kola and the film former on to the nail.

2. The composition of claim 1 wherein the gotu kola is substantially between 0.0001 to 2% by weight based upon the total weight of the composition.

3. The composition of claim 1 wherein the gotu kola is substantially between 0.00001 to 2% by weight based upon the total weight of the composition.

4. The composition of claim 1 further comprising a plasticizer.

5. The composition of claim 1 further comprising an ultra-violet radiation absorber.

6. The composition of claim 1 further comprising at least one pigment.

7. The composition of claim 1 further comprising a viscosity controlling agent.

8. The composition of claim 1 further comprising a vitamin.

9. A topical nail application composition comprising:
a nail lacquer including a film former dissolved in a solvent and at least one additional component selected from the group consisting of a plasticizer, a viscosity controlling agent, a ultra violet absorbers, and a pigment; and
gotu kola.

10. A nail treatment application having a total weight comprising gotu kola.

11. A nail treatment as claimed in claim 10 wherein gotu kola is substantially between 0.0001 to 2% by weight based upon the total weight of the nail treatment.
